(19) United States
(12) Patent Application Publication
(10) Pub. No.: US 2016/0296206 A1
(43) Pub. Date: Oct. 13, 2016

(54) OBJECT INFORMATION ACQUIRING APPARATUS

(71) Applicant: CANON KABUSHIKI KAISHA, Tokyo (JP)

(72) Inventors: Hisafumi Ebisawa, Tokyo (JP); Kenichi Nagae, Yokohama-shi (JP)

(21) Appl. No.: 15/093,877

(22) Filed: Apr. 8, 2016

(30) Foreign Application Priority Data
Apr. 9, 2015 (JP) 2015-079845

(51) Int. Cl. A61B 8/00; A61B 5/00; A61B 5/145; A61B 8/08

(52) U.S. Cl. CPC A61B 8/4461; A61B 8/5207; A61B 8/0825; A61B 8/40; A61B 5/0095; A61B 5/14546; A61B 5/14542; A61B 5/4872; A61B 5/4875; A61B 8/483; A61B 8/466

(57) ABSTRACT

An object information acquiring apparatus comprising: a receiver in which a plurality of elements is arranged, each of the plurality of elements receives an acoustic wave propagating from an object and outputs an electric signal; a scanner configured to move the receiver through a predetermined scanning region; an information processor configured to acquire characteristics information on an interior of the object using the electric signal; and an acoustic attenuator configured to be arranged between the receiver and the object in the scanning region and have an acoustic attenuation characteristics distribution corresponding to a shape of the object is used.

### BACKGROUND OF THE INVENTION

1. Field of the Invention

The present invention relates to an object information acquiring apparatus.

2. Description of the Related Art

Research of object information acquiring apparatuses using ultrasonic waves has been promoted in order to obtain characteristics information on the interior of an object such as the breasts. For example, ultrasonic apparatuses are available which receive an echo signal reflected by the object to generate characteristics information, and photoacoustic apparatuses are also available which irradiate the object with laser light to receive an ultrasonic wave (photoacoustic wave) resulting from a photoacoustic effect to generate characteristics information.

In the ultrasonic apparatus in Japanese Patent Application Laid-open No. 2008-073305, a probe arranged on a floor portion of a tank obtains three-dimensional image data by transmitting and receiving an ultrasonic wave to and from the breasts suspended and immersed in the water tank while mechanically moving in a horizontal plane. The resultant data can be displayed on a monitor, for example, as any desired sectional images of the breasts.

Patent Literature 1: Japanese Patent Application Laid-open No. 2008-073305

### SUMMARY OF THE INVENTION

A direction in which the probe transmits the ultrasonic wave is referred to as a “depth”. The probe in Japanese Patent Application Laid-open No. 2008-073305 performs scans in the horizontal plane, and thus, a distance from the probe to a surface of each of the breasts varies between a case where the probe opposes a tip portion (central portion) of the breast and a case where the probe opposes a peripheral portion of the breast. Therefore, the ratio between water and the body tissue in a path of the ultrasonic wave varies between the tip portion and the peripheral portion of the breast. In general, the body tissue is more likely to attenuate the ultrasonic wave than water.

Thus, under the same measurement conditions, the ultrasonic wave traveling from the probe to a depth L has a lower intensity when the probe opposes the tip portion of the breast than when the probe opposes the peripheral portion of the breast. Similarly, the ultrasonic wave traveling from the position of the depth L to the probe has a lower intensity in the case of the tip portion than in the case of the peripheral portion. As a result, in a sectional image such as a C plane image which is parallel to a scanning plane (for example, an image at the depth L), a high intensity (bright color) is obtained in the peripheral portion, while a low intensity (dark color) is obtained in the tip portion. Such a decrease may lead to a reduced contrast in display images or a reduced accuracy of image analysis.

In the case of Japanese Patent Application Laid-open No. 2008-073305, a difference in acoustic attenuation characteristics is caused during both transmission and reception of the ultrasonic wave. However, this problem also occurs in a form in which only the transmission or the reception is performed. For example, even when, at the same depth L in the breast to be measured by the photoacoustic apparatus, ultrasonic waves (photoacoustic waves) with the same intensity are generated in the peripheral portion and in the tip portion, the intensity of the signal reaching the probe varies.

The present invention has been developed in view of the above-described problems. An object of the present invention is to provide a technique for an apparatus that acquires characteristics information on an object by allowing a probe to scan the object, while receiving an ultrasonic wave from the object, the technique dealing with changes in the degree of attenuation according to the position of the probe.

The present invention provides an object information acquiring apparatus comprising:
a receiver in which a plurality of elements is arranged, each of the plurality of elements receives an acoustic wave propagating from an object and outputs an electric signal;
a scanner configured to move the receiver through a predetermined scanning region;
an information processor configured to acquire characteristics information on an interior of the object using the electric signal; and
an acoustic attenuator configured to be arranged between the receiver and the object in the scanning region and have an acoustic attenuation characteristics distribution corresponding to a shape of the object

The present invention can provide a technique for an apparatus that acquires characteristics information on an object by allowing a probe to scan the object, while receiving an ultrasonic wave from the object, the technique dealing with changes in the degree of attenuation according to the position of the probe.

Further features of the present invention will become apparent from the following description of exemplary embodiments with reference to the attached drawings.

### BRIEF DESCRIPTION OF THE DRAWINGS

FIG. 1 is diagram depicting a configuration of an object information acquiring apparatus in Embodiment 1;

FIG. 2 is a diagram depicting a configuration of a signal processor;

FIG. 3 is a diagram depicting a distance between a probe and an object and an object thickness from the object surface to any C plane;

FIG. 4 is a diagram depicting a form in which a single member is used as a holding member to provide a distribution of a film thickness;

FIGS. 5A, 5B, and 5C are diagrams depicting formations of the holding member;

FIG. 6 is a diagram depicting a form in which a different member is used as the holding member;

FIGS. 7A, 7B, and 7C are diagrams depicting formations of the holding member;

FIGS. 8A, 8B, and 8C are diagrams illustrating the use of a convex probe and bowl-shaped probes;

FIGS. 9A and 9B are diagrams depicting a configuration of an object information acquiring apparatus in Embodiment 2; and

FIG. 10 is a diagram depicting a configuration of an object information acquiring apparatus in Embodiment 5.

### DESCRIPTION OF THE EMBODIMENTS

Preferred embodiments of the present invention will be described below with reference to the drawings.

However, dimensions, materials, shapes, relative arrangements, and the like of components described below should be changed as needed according to a configuration of an apparatus to which the present invention is applied and various conditions. Hence, the dimensions, materials, shapes, relative arrangements, and the like of the components described below are not intended to limit the scope of the present invention to the following description.

The present invention relates to a technique for detecting an acoustic wave propagating from an object to generate and acquire characteristics information on the interior of the object. Hence, the present invention may be considered as an object information acquiring apparatus or a method for controlling the object information acquiring apparatus, or an object information acquiring method or a signal processing method. The present invention may also be considered as a program that allows an information processing apparatus including hardware resources such as a CPU to execute these methods or a storage medium storing such a program.

An object information acquiring apparatus in the present invention includes an apparatus that uses a photoacoustic tomography technique to irradiate an object with light (electromagnetic wave) and receive (detect) an acoustic wave propagating after being generated at a specific position in the object or on a surface of the object, in accordance with a photoacoustic effect. Such an object information acquiring apparatus obtains characteristics information on the interior of the object in the form of image data based on photoacoustic measurement, hence the apparatus can be called a photoacoustic imaging apparatus.

The characteristics information in the photoacoustic apparatus refers to a source distribution for acoustic waves resulting from light irradiation, an initial sound pressure distribution in the object, or an optical-energy absorption density distribution or an absorption coefficient distribution derived from the initial sound pressure distribution, or a concentration distribution for substances constituting a tissue. A specific example of the characteristics information includes an oxidized- and reduced-hemoglobin concentration distribution, an oxygen saturation distribution or a blood component distribution derived from the oxidized- and reduced-hemoglobin concentration distribution, or a distribution of fat, collagen, mammary gland, or moisture. The characteristics information may be obtained in the form of distribution information on positions in the object instead of numerical data. That is, the object information may be distribution information such as absorption coefficient distribution or an oxygen saturation distribution.

The object information acquiring apparatus in the present invention includes an apparatus that utilizes an ultrasonic echo technique and that transmits an ultrasonic wave to an object and that receives a reflected wave (echo wave) reflected inside the object to acquire object information in the form of image data. For the apparatus utilizing the ultrasonic echo technique, the object information acquired is information reflecting differences in acoustic impedance among tissues inside the object.

The acoustic wave referred to in the present invention is typically an ultrasonic wave and includes an elastic wave referred to as a sound wave or an acoustic wave. An acoustic wave resulting from a photoacoustic effect is referred to as a photoacoustic wave or an optical ultrasonic wave. An electric signal into which an acoustic wave is converted by a probe or the like is also referred to as an acoustic wave. However, the ultrasonic wave or the acoustic wave referred to herein is not intended to limit the wavelength of an elastic wave. An electric signal based on a photoacoustic wave is also referred to as a photoacoustic wave. An electric signal based on an ultrasonic echo is also referred to as an ultrasonic signal.

### EMBODIMENT 1

### Apparatus Configuration

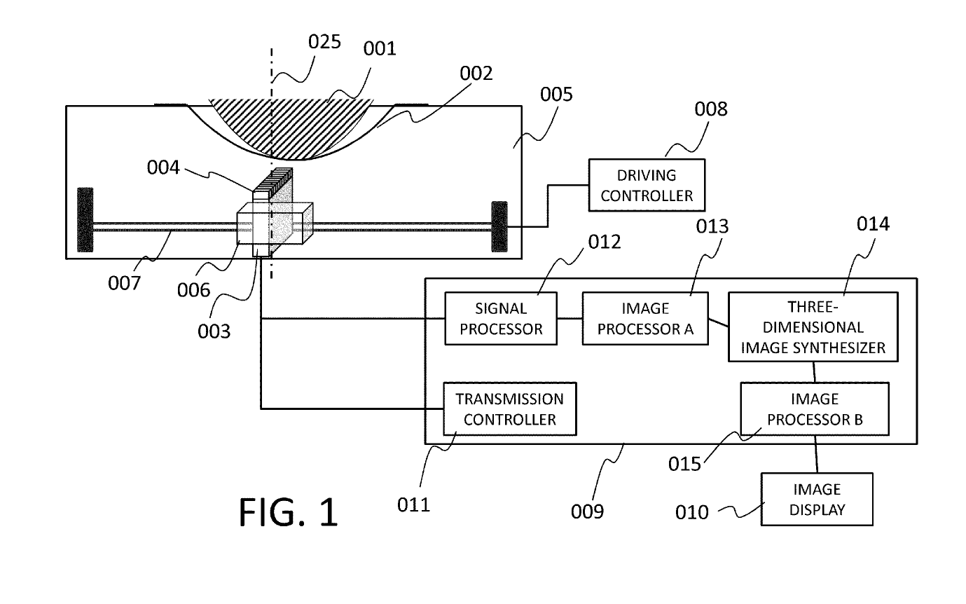

With reference to FIG. 1, a configuration example will be described in which the present invention is applied to an ultrasonic echo apparatus. An object (for example, the breast) is denoted by reference numeral 001. A holding member that holds the object 001 is denoted by reference numeral 002. A probe that transmits an ultrasonic wave and detects an echo wave from the interior of the object is denoted by reference numeral 003. The probe 003 has a plurality of conversion elements 004. An acoustic matching material 005 through which an acoustic wave propagates is present between the probe 003 and the holding member 002. The probe 003 is fixed on a carriage 006. The carriage 006 is moved by a driving mechanism 007. A driving controller 008 serves to control the driving mechanism 007.

A system controller 009 creates a three-dimensional image from an image signal for the object 001 received by the probe 003 within a scan range. An image display 010 displays the three-dimensional image created by the system controller 009. The probe corresponds to a receiver in the present invention. The holding member corresponds to a holding member in the present invention. The driving mechanism corresponds to a scanner in the present invention. The system controller corresponds to an information processor in the present invention.

The system controller 009 includes a plurality of units 011-015, any or all of which may be implemented by a computer, ASIC, etc. A transmission controller 011 controls a driving timing for each of the conversion elements 004 corresponding to a focus position in order to adjust a transmission focus of an ultrasonic wave. A signal processor 012 reconstructs an ultrasonic echo signal from the object 001 into a two-dimensional image. An image processor A (013) executes image processing on the reconstructed image data. A three-dimensional image synthesizer 014 converts the reconstructed image into a three-dimensional image based on coordinates of the probe 003 driven for scanning by the driving mechanism 007 to perform scanning. An image processor B (015) executes image processing on the three-dimensional image data.

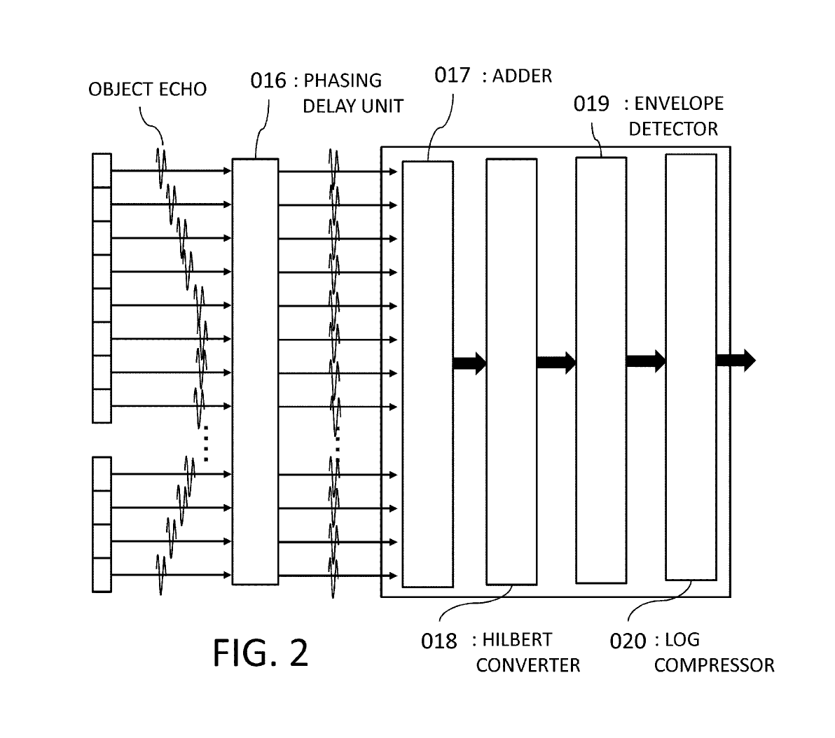

FIG. 2 depicts a configuration of the signal processor 012. A phasing delay unit 016 adjusts phases of signals received by the conversion elements 004. An adder 017 adds the delayed signals together. A Hilbert converter 018 executes Hilbert conversion on a signal resulting from the addition. An envelope detector 019 performs detection. An LOG compressor 020 performs LOG compression on the detected signal. This signal processor is an example and may have any configuration as long as the signal processor can perform amplification, digital conversion, correction, delay, or the like on electric signals output from the conversion elements.

### Ultrasonic Wave Transmission and Reception and Image Reconstruction by the System Controller

The system controller transmits an ultrasonic wave to the object 001 and converts an echo signal generated inside the object or on the surface of the object. The transmission controller 011 determines a delay time according to which a plurality of group of the conversion elements 004 forming a transmission aperture are driven in order to focus a transmission beam at a desired position (a position with respect to the probe in a ultrasonic transmission direction, that is, a depth). The transmission controller 011 sends driving signals to the conversion elements 004 based on the delay time. Then, the conversion elements 004 generate ultrasonic waves, based on the driving signals and transmit the ultrasonic wave to the object 001.

The transmitted ultrasonic waves propagate through the acoustic matching material 005 and the holding member 002 to the object 001. Subsequently, echo waves reflected and scattered by the object 001 partly return to the conversion elements 004. A plurality of groups of conversion elements 004 forming a reception aperture receive and convert the echo waves into electric signals (reception signals). Amplification, correction, digital conversion, or the like is executed on the reception signals as needed.

The reception signals are reconstructed by the signal processor 012 into image data indicative of characteristics information. In FIG. 2, the phasing delay unit 016 determines a delay time for reception signals based on an imaging position on an image scan line 025 in FIG. 1 and coordinate information on the positions of the conversion elements 004 forming the reception aperture. The phasing delay unit 016 then executes a delay process on the reception signals.

The delayed reception signals are added together by the adder 017. Subsequently, a resultant synthetic signal is subjected to Hilbert conversion and envelope detection by the Hilbert converter 018 and the envelope detector 019 to reconstruct an image. Besides the phasing addition method described herein, a reconstruction technique such as adaptive signal processing may be utilized. The reconstructed image data undergoes LOG compression by the LOG compressor 020 to complete image data on the image scan line 025. A series of processes is executed with the image scan line 025 moved to create a two-dimensional ultrasonic image data along a scan direction.

The image processor A (013) executes an edge emphasis process, a noise removal process, a contrast emphasis process, or the like on the created two-dimensional ultrasonic image data. These types of image processing may be implemented by the image processor B (015). The system controller executes the above-described processing on data obtained by the probe 003 transmitting and receiving ultrasonic waves while moving in a scanning region to accumulate two-dimensional image data to generate three-dimensional image data. After executing the three-dimensional-image acquisition process, the three-dimensional image synthesizer 014 arranges the three-dimensional image data in association with coordinate positions in the scanning region defined by the driving controller 008. The shape of the scanning region is not limited to a general plane. The driving controller may move the probe in three-dimensional directions.

Instead of being executed after a B mode image is created, the three-dimensional-image acquisition process may be achieved by using the signal processor 012 to accumulate signals without implementing processes subsequent to the process by the Hilbert converter 018 and using the three-dimensional image synthesizer 014 to execute a synthetic aperture process. The synthetic aperture process allows the resolution of images in a scanning direction of the probe 003 to be uniformized in the depth direction. Various other known techniques for obtaining three-dimensional image synthesizer 014 may be utilized.

The image processor B (015) adjusts the created three-dimensional image data, for example, executes a sharpening process, a noise removal process, or the like. The image display 010 displays any desired sectional images. Image processing can be used to reduce brightness unevenness at the same depth that results from a variation in the degree of attenuation and that is a problem to be solved by the present invention. However, eliminating loss of image information is impossible. As the image display 010, a liquid crystal display, a plasma display, an organic EL display, or the like may be utilized. The image display 010 need not necessarily be a part of the apparatus. It is also preferable that the apparatus in the present invention create only image data and allow an external image display to display the image data.

### Driving of the Probe

The probe 003 will be described. Each of the conversion elements 004 performs conversion between an electric signal and an ultrasonic wave. Preferred conversion elements are piezoelectric elements such as PZTs, PVDF elements, cMUT elements, and the like which have relatively high conversion efficiency. The use of a probe with a plurality of conversion elements 004 one- or two-dimensionally arranged therein is expected to improve an SN ratio and to reduce measurement time.

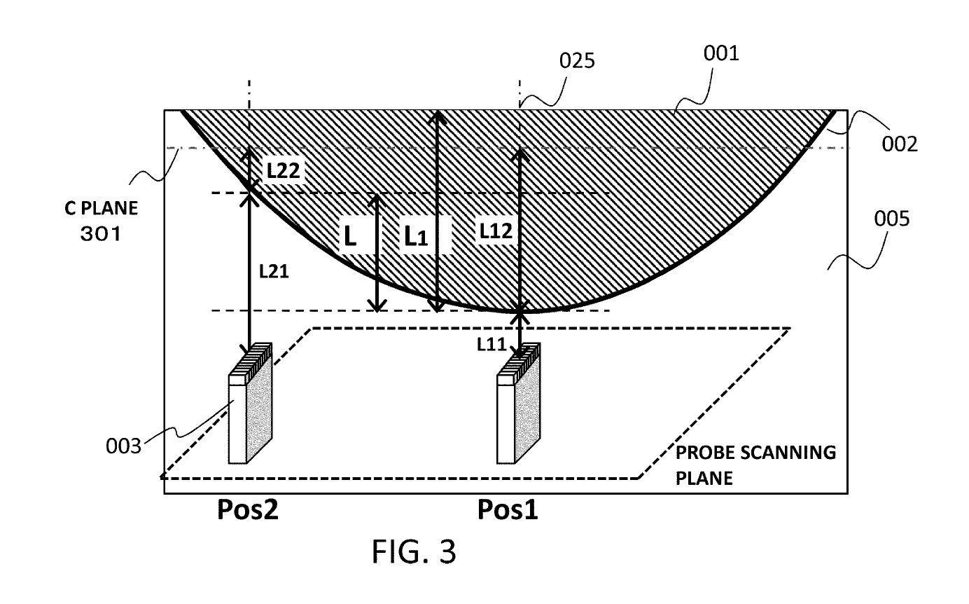

Driving of the probe 003 and an imaging method used during the driving will be described with reference to FIG. 3. The probe 003 installed on the carriage 006 is moved by the driving mechanism 007 in a two-dimensional plane opposite to the holding member 002. As the driving mechanism 007, for example, a combination of a pulse motor and a ball screw or a linear motor may be utilized. The driving mechanism 007 may drive the probe 003 in the three-dimensional directions. A rotation mechanism for the carriage 006 may be provided to tilt the probe 003 to any angle. The three-dimensional movement and tilt of the probe allows ultrasonic waves to be obtained in various directions with respect to the object, whereby accurate image data can be obtained.

### Holding Member

The use of the holding member 002 stabilizes the shape of the object to improve the calculation accuracy of calculations of the degree of attenuation and calculations for image reconstruction. The holding member 002 used is transmissive for acoustic waves. A material for the holding member 002 desirably involves a small difference in acoustic impedance between the object 001 and the acoustic matching material 005. In order to allow the object 001 to be held, a rigid member or a stretchable member is preferably used. Examples of the rigid member include resin materials such as PET, polymethyl pentene, and acrylic. Examples of the stretchable member include rubber sheets of latex, silicone, and the like and materials such as urethane. Alternatively, a holding mechanism containing a combination of a plurality of materials may be used.

Preferably, the holding member 002 is interchangeably installed. When the breast is inserted into the apparatus through an opening in a housing, an installation portion may be provided in the vicinity of the opening, with this portion including a bracket or a hook to allow the holding member to be easily fixed. This allows the holding member 002 to be easily replaced with according to the subject or according to what or how to measure the object.

The acoustic matching material 005 acoustically matches the object (or the holding member) with the probe. Therefore, the acoustic matching material 005 preferably allows acoustic waves to propagate through the acoustic matching material 005 and avoids preventing scanning by the probe 003. Examples of the acoustic matching material 005 include liquids such as water, DIDS, PEG, silicone oil, and castor oil.

In a case where the object is a living organism, there are many regions with curvatures or many uneven shapes. A breast, for example, has a central portion protruding with respect to a peripheral portion. In contrast, the object may have a shape such that the central portion is depressed with respect to the peripheral portion as in the case of buttocks and the arch of a foot. As a result, a distance between a probe scanning plane and the object in a direction normal to the scanning plane (or a high-sensitivity direction of the conversion elements) varies according to the position of the probe.

### Positional Relation between the Holding Member and the Object Surface

In an example in FIG. 3, the scanning plane of the probe 003 is also not parallel to the surface of the object 001. In FIG. 3, a C plane 301 is to be displayed. When the probe scanning plane is parallel to the C plane and the probe is located at Pos1, a distance L11 is present from the probe to the object surface, and a distance L12 is present from the object surface to the C plane. When the probe is at Pos2, a distance L21 is present from the probe to the object surface, and a distance L22 is present from the object surface to the C plane. Thus, the ratio between an in vivo passage distance and an acoustic matching material passage distance on the path of an ultrasonic wave varies between the case where the probe 003 is located at Pos1 and the case where the probe 003 is located at Pos2. In general, an ultrasonic wave attenuation rate is higher in the living organism than in the acoustic matching material. Consequently, both the transmitted ultrasonic wave and the echo wave are more likely to attenuate at Pos1 than at Pos2. As a result, the value of brightness in image data varies.

When there is a large difference in brightness within the same C plane, the degree of reproduction of images of the interior of the object may decrease in image display, particularly in real-time display. For example, assume that the image data contained in certain C plane image data has a brightness varying between “0 and 100”. If an operator adjusts the range of the brightness of images displayed on the display to between “20 and 80”, information is lost which concerns pixels with brightness values falling outside the range. Therefore, particularly in ultrasonic apparatuses that display images in real time, the accuracy of image analysis may decrease.

Such problems caused by a variation in output value will be described below in further detail. For example, in image data at a C plane position corresponding to Pos1, the output value is multiplied by a larger gain in order to correct attenuation during in vivo propagation over a long distance. However, application of this condition to Post may lead to an excessive gain to the output value. Specifically, amplification is performed by the value of the product of three numerical values—a distance difference L, the acoustic attenuation characteristics of the object 001, and an ultrasonic frequency during transmission and reception. As a result, the gain reaches several tens of dB depending on conditions and may exceed an upper limit of a dynamic range of display brightness. In contrast, when the image data at the C plane position corresponding to Pos1 is imaged under conditions set to allow image data at a C plane position corresponding to Post to be displayed, the amplification may be insufficient and signal intensity may be lower than the noise level of the apparatus.

### Preferred Acoustic Attenuation Characteristic of the Holding Member

To avoid this phenomenon, an acoustic attenuation difference equivalent to the distance difference L needs to be compensated for. In this case, the acoustic attenuation difference is compensated for by distributing the acoustic attenuation characteristics in the holding member 002. Specifically, a difference in acoustic attenuation characteristics between the object 001 and the acoustic matching material 005 for the length L [cm] is reflected in the acoustic attenuation characteristics of the holding member 002. For example, as the acoustic matching material 005, water is used, which hardly attenuates acoustic waves. At this time, when the attenuation characteristic of the object 001 is assumed to be 0.3 [dB/MHz/cm], a difference of 0.3 L [dB/MHz] occurs between the degree of attenuation at Post and the degree of attenuation at Pos2. Thus, the output value difference at the C plane can be reduced by adjusting, in the holding member 002, the difference between the acoustic attenuation characteristics at the position corresponding to Pos1 and the acoustic attenuation characteristics at the position corresponding to Pos2 to 0.3 L [dB/MHz].

Such adjustment is made as needed in accordance with the difference in acoustic attenuation characteristics between the object 001 and the acoustic matching material 005 and the distance that acoustic waves propagate through the object. In general, the object 001 has a higher level of acoustic attenuation characteristics, and thus, it is preferable to reduce the level of the acoustic attenuation characteristics of the holding member 002 corresponding to a location where the probe 003 and the object 001 are in close proximity to each other.

Due to the characteristics of human bodies, many objects 001 are round in shape and are likely to protrude at a central portion when held. Thus, the level of the acoustic attenuation characteristics of the holding member 002 can be effectively adjusted to be lower at the central portion than at the peripheral portion. More specifically, when the scanning plane is generally planar, the degree of acoustic attenuation increases consistently with distance from the scanning plane to the holding member in a direction normal to the scanning plane. In contrast, the degree of acoustic attenuation decreases consistently with distance from the scanning plane to the holding member in the direction normal to the scanning plane.

Calculation of the acoustic attenuation characteristics needs the following four pieces of information.
(Information 1) acoustic attenuation characteristics of the acoustic matching material 005: α2 [dB/MHz/cm]
(Information 2) acoustic attenuation characteristics of the object 001: α1 [dB/MHz/cm]
(Information 3) holding shape of the object 001
(Information 4) scanning trajectory of the probe 003

The information 1 and the information 4 are known from settings for the system and materials used. On the other hand, the information 2 and the information 3 involve significant variations among tissues and large differences among individuals and are thus preferably set with reference to experimental values and literature values. The holding member 002 can be effectively produced or selected based on object information acquired through prescans.

The information 2 is preferably specified such that, when the object 001 is the breast, α1 =0.3 to 0.8 [B/mHz/cm]. The breast is characterized in that young people tend to have many mammary gland layers and that the rate of fat tends to increase with age. The mammary gland layer has a higher level of acoustic attenuation characteristics than the fat layer, and thus, the level of the acoustic attenuation characteristics of the breast is preferably increased with decreasing age.

When the object 001 is a soft tissue such as the breast, a rigid member is used as the holding member in order to accurately obtain the information 3 (holding shape). The shape of the holding member 002 preferably fits the object 001. For example, for the breast, the holding member 002 is shaped like a cup. The rigid member allows the holding shape of the object 001 to be prescribed, whereby the distance between the probe 003 and the object 001 is also prescribed. This makes it easier to obtain the information 3.

On the other hand, when a stretchable material is selected as the holding member, strictly defining the holding shape is difficult. However, the holding shape can be estimated to some degree based on the hardness and film thickness of the member, information on the object 001, and the like. The information on the object 001 includes, for example, for the breast, size information such as a cup size, a topbust size, and an underbust size, and other information such as race, age, and body conditions. Specifically, the amount of protrusion (L1) of the object 001 can be suppressed by increasing the hardness of the holding member 002, increasing the film thickness, or pre-tensioning the holding member 002. When the object 001 is the breast, the breast is difficult to squeeze when the subject is young and has many mammary gland layers or when the subject has menstruation. These pieces of information are used to customize the holding member 002 to allow the holding shape to be specified in detail, reducing the non-uniformity of output values obtained from any desired section.

The above-described four items allow an acoustic attenuation characteristics difference to be specified for each position of the probe 003. The maximum value of the acoustic attenuation characteristics difference depending on the position can be converted using the following expression.

(α1−α2)×L1 (1)

In this case, the acoustic attenuation characteristics α1 of the object 001 is assumed to have a value of 0.3 to 0.8 [dB/MHz/cm], and the maximum L distance is assumed to have a value of 1 to 9 [cm]. For the breast, the maximum L distance refers to the amount of protrusion L1 over the maximum distance from the tip of the breast to the chest wall. The acoustic matching material 005 is assumed to be water (α2 is 0 [dB/MHz/cm]). When the assumptions are applied to the above-described expression, the preferred acoustic attenuation characteristics difference falls within the range of 0.3 to 7.2 [dB/MHz].

### Production Method 1 for the Holding Member: Adjustment of the Film Thickness

Now, a technique for producing the holding member 002 with the desired acoustic attenuation characteristics distribution will be described. First, a technique is available which involves using a single material as the material for the holding member 002 and varying the film thickness for each segment to adjust the acoustic attenuation characteristics. For the single material, the acoustic attenuation characteristics changes in proportion to the film thickness. Thus, higher machining accuracy for the holding member 002 allows the acoustic attenuation characteristics to be adjusted in more detailed manner.

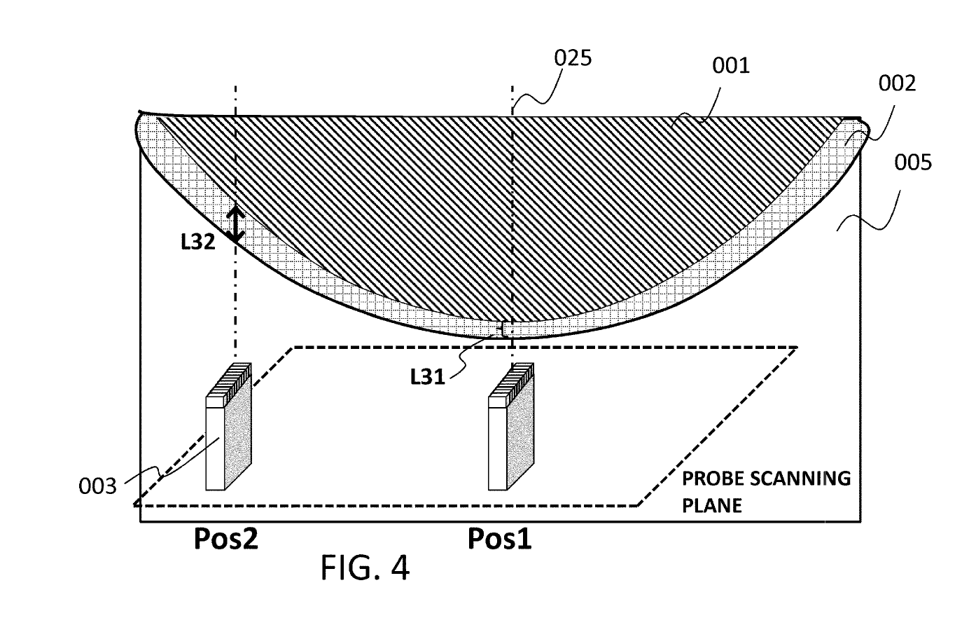

In FIG. 4, the film thickness means the length of an internal path in the holding member 002 on the image scan line 025 in a holding status. Thus, the film thickness also changes according to the inclination angle of the holding member 002. The maximum difference in the thickness in the preferred holding member 002 can be converted using the following expression.

(α1−α2)×L1/α3 [cm] (2)

When a latex rubber sheet (having an acoustic attenuation characteristics α3 of approximately 5 [dB/MHz/cm]) is used as the holding member 002, the maximum difference in the thickness in the holding member 002 preferably falls within the range of 0.6 to 14.4 [mm].

When the object 001 has a higher level of acoustic attenuation characteristics than the acoustic matching material 005 and the object 001 protrudes at the central portion thereof, as shown in FIG. 4, the film thickness of the holding member 002 is preferably made thinner at the central portion than at the peripheral portion (L31<L32). The holding member 002 is preferably produced by compression processing or ejection molding using a specified mold. A manufacturing method such as machining can be utilized or a 3D printer may be used to produce the holding member 002. The acoustic attenuation characteristics can be adjusted in detail by enabling the holding member 002 to be molded into any shape.

### Production Method 2 for the Holding Member: Combination of Sheet Members

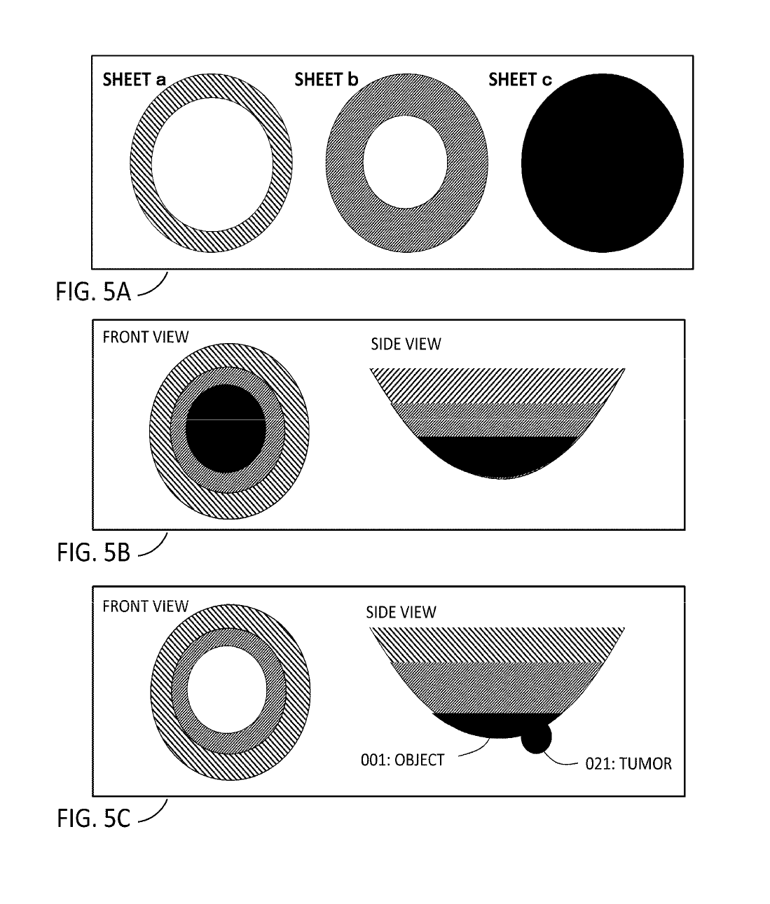

Another method is available in which the film thickness is distributed by combining a plurality of sheet members together. Specifically, a plurality of sheets (a) to (c) with different hole diameters as depicted in FIG. 5A is prepared and laid on top of one another as depicted in FIG. 5B. Any method is used to lay the sheets on top of one another. First, a method is available which uses an adhesive having an acoustic impedance close to the acoustic impedance of the material of the sheets. For example, when the member is silicone rubber, a silicone-based adhesive is suitable. In the description below, a front view refers to a view depicting the holding member as seen from the probe side. A side view refers to a view depicting the holding member as seen from a side of the apparatus.

Alternatively, the members may be simply laid on top of one another. In particular, a rubber member and a gel member have a self melt adhesion property, and thus allows the holding member to be formed without the use of any adhesive. When this method is used, spaces between the sheet members are preferably filled with the acoustic matching material 005 with an acoustic impedance close to the acoustic impedance of the sheet members to prevent the entry of air bubbles. This applies to, for example, a case where the sheet member and the acoustic matching material 005 are latex and water, respectively, and a case where the sheet member and the acoustic matching material 005 are silicone rubber and silicone oil, respectively. If the sheet members have significantly different acoustic impedances or air bubbles enter between the sheet members, ultrasonic waves are reflected by the interface between the sheet members, causing an artifact.

These methods for combining a plurality of members have the advantage of facilitating customization of the holding member 002 in accordance with the situation of the subject or the object 001. For example, when the object 001 is large, a large number of sheet members are combined together to increase the difference in film thickness between the central portion and the peripheral portion. If a large tumor 021 is present in the central portion, only ring-like sheets each with a hole formed at the center as depicted in FIG. 5C (corresponding to the sheet a and the sheet b in FIG. 5A) are combined together to effectively hold the object at the peripheral portion thereof.

When the film thickness is adjusted or the sheet members are laid on top of one another to increase the film thickness of the holding member 002 at the peripheral portion thereof, not only can a variation in image output value be suppressed but the mechanical strength of the holding member 002 and a force to hold the object 001 can be increased. In general, the force with which the object 001 presses the holding member 002 is several tens of N or more, hence the film thickness of the holding member 002 at the peripheral portion thereof is preferably relatively large in order to strengthen the peripheral portion. When the holding member 002 is a stretchable member, the film thickness of the holding member 002 at the peripheral portion thereof is also preferably relatively large in order to stably hold the object even when the body moves during imaging. Consequently, distortion of images and degradation of resolution can be suppressed.

### Production Method 3 for the Holding Member: Combination of a Plurality of Materials

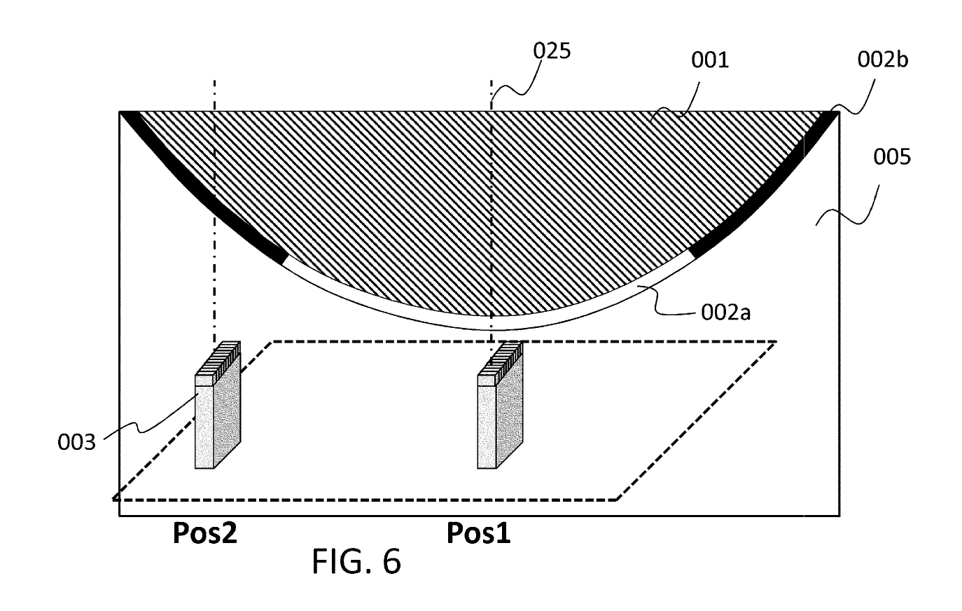

Using FIG. 6, a technique will be described in which a plurality of materials with different acoustic attenuation characteristics is combined together to distribute the acoustic attenuation characteristics in the plane of the holding member 002. For example, when the object protrudes at the central portion thereof and is more likely to attenuate acoustic waves than the acoustic matching material, a member with a low degree of attenuation is used for the central portion of the holding member (reference numeral 002a in FIG. 6), while a member with a high degree of attenuation is used for the peripheral portion of the holding member (reference numeral 002b in FIG. 6).

For example, for the rigid member, acrylic or polymethyl pentene, which has relatively insignificant acoustic attenuation characteristics, is suitable for the central portion, while PET material or the like, which has relatively high acoustic attenuation characteristics, is suitable for the peripheral portion. For the stretchable member, silicone rubber, which has relatively insignificant acoustic attenuation characteristics, is suitable for the central portion, while latex rubber, which has relatively significant acoustic attenuation characteristics, is suitable for the peripheral portion. Even the same raw material exhibits different acoustic attenuation characteristics depending on the composition of the material. For example, the composition ratio of a urethane raw material is changed to allow production of members with different attenuation characteristics. In this technique, as is the case with Production Method 1, the acoustic attenuation characteristics are preferably adjusted by fine-tuning the film thickness for each segment or laying the members on top of one another as in the case of Production Method 2. The material is not limited to the two types. For example, a concentric and circular configuration may be used in which the acoustic attenuation characteristics become gradually more significant from an innermost circle toward an outermost circle.

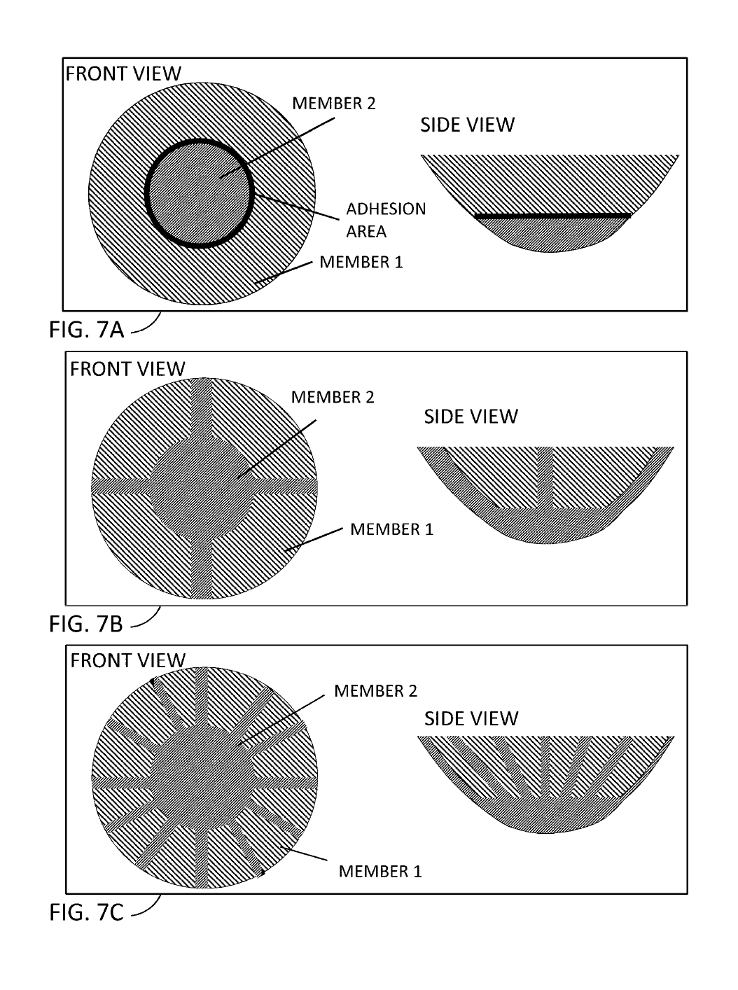

Examples of Production Method 3 will be described using FIGS. 7A, 7B, and 7C. The figures are front views and side views of the holding member. In FIG. 7A, a ring-shaped member 1 and a circular member 2 that is equal in size to a hole in the ring are prepared and coupled together by adhesion. That is, on a adhesion surface, a plurality of films is laid on top of one another and the adhesive is present, making the acoustic attenuation characteristics likely to change. That is, the difference in acoustic impedance between the adhesive and the member may cause an artifact. Thus, preferably, the amount of the adhesive is reduced or an adhesive is used which is only slightly different in acoustic impedance from the member. Preferably, the adhesion surface is reduced in size or the member thickness of the adhesion surface is reduced. When the member is a rubber material, the holding member may be formed utilizing the self melt adhesion property. However, in this case, it should be noted that air bubbles may enter the member.

FIG. 7B depicts a combination of a ring-shaped member 1, a circular member that is equal in size to a hole in the ring, and a shaped member 2 with branch portions radially extending from the circular area toward the periphery. In this case, the holding member can advantageously be formed without the use of any adhesive. However, the acoustic attenuation may be uneven at the branch portions, and thus, the area of the branch portions is preferably reduced. However, the area of the branch portions is determined based on the balance between the strength and the acoustic characteristics of the holding member.

Alternatively, instead of reducing the area per branch portion, preferably more branch portions are arranged at regular intervals throughout the plane as depicted in FIG. 7C. For transmission and reception of ultrasonic waves, an aperture width is present. A plurality of sound rays forming an image passes through the holding member 002 at different positions. Thus, the branch portions are distributively positioned as depicted in FIG. 7C to allow constant rates of sound rays to pass through the holding member 002. This enables a reduction in image unevenness near the branch portions.

Even for a holding member formed of the same raw material, an acoustic attenuation characteristics distribution can be formed by varying the composition of the holding member with segment. For example, a method is available in which the hardness is adjusted by adding a hardener to urethane or a rubber member. For example, addition of the hardener allows urethane to be adjusted to have varying hardness ranging from a high hardness like that of plastic to softness like that of rubber. The acoustic attenuation characteristics vary according to the hardness. The level of the acoustic attenuation characteristics increases consistently with the size of an ultrasonic scattering medium added to the member. A hardener that is compatible with the base material may be used.

Moreover, a method is available which involves adjusting the amount of the ultrasonic scattering medium added to the member. For example, glass beads with a diameter of micron order are mixed into a urethane gel to allow the acoustic attenuation characteristics to be adjusted. As the ultrasonic scattering medium, titanium oxide or silica may be used besides the glass beads. Different scattering media may be added to the holding member in association with segments in the holding member.

### Configuration of the Image Processor

The difference among output values from any desired section can be reduced by changing the acoustic attenuation characteristics in the plane of the holding member 002 as described above. The image processor B (reference numeral 015) may adjust the output values to further reduce the output value difference to allow brightness unevenness to be suppressed.

The above description is based on the ultrasonic image apparatus that irradiates the object with an ultrasonic wave, receives an echo signal and converts the same into an image. However, the above-described configuration may be applied to a photoacoustic image apparatus that receives and images an ultrasonic wave generated by the object 001 irradiated with light from a light source.

In the above-described method, when the degree of acoustic attenuation is varied between a protruding portion and a depressed portion of the object, the brightness in the same C plane is suitably displayed, whereas display brightness may change as a result of reconstruction in spite of an equivalent initial sound pressure. Specifically, when the initial sound pressure intensity is reconstructed at a depth of 1 cm from the object surface on a scan line at Pos1 and on a scan line at Post, a higher initial sound pressure intensity is displayed at Pos1, where the degree of attenuation is lower. Thus, when any plane other than the C plane is displayed, the intensity of the reception signal is preferably multiplied by such a gain as compensates for the acoustic attenuation characteristics distribution.

### Variation of the Probe

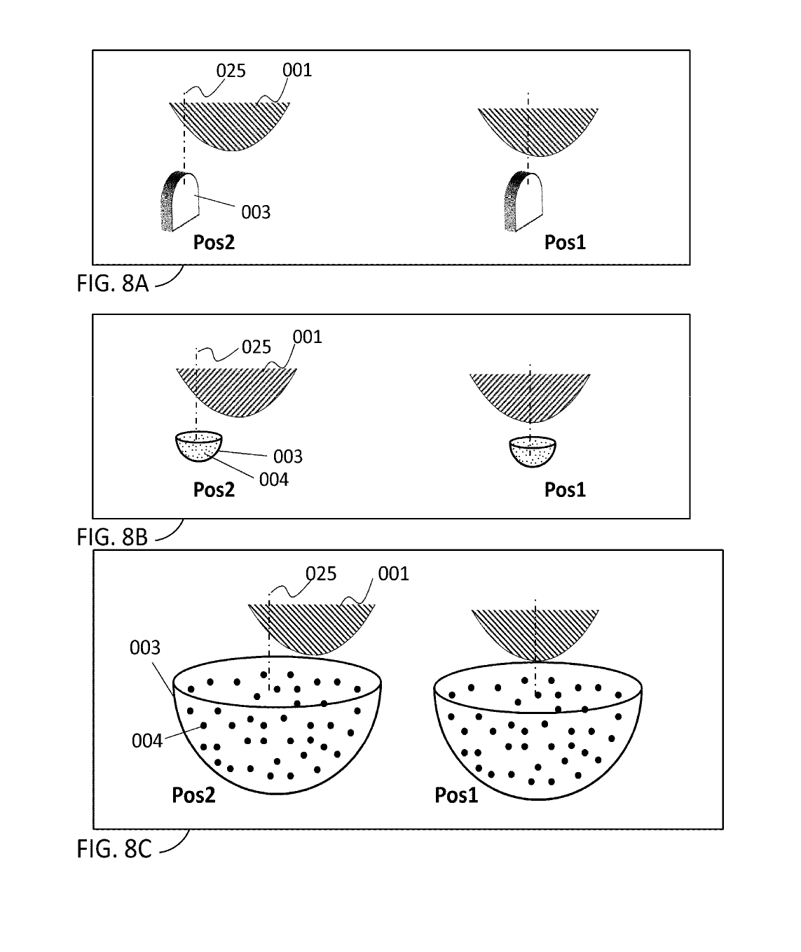

The present invention is applicable to an apparatus including any of various such probes as depicted in FIGS. 8A, 8B, and 8C instead of a 1D probe or a 2D probe. For example, FIG. 8A depicts a convex probe with the conversion elements 004 arranged therein so as to have a curvature. FIGS. 8B and 8C depict a large and a small bowl-shaped probes with conversion elements arranged on a hemispherical surface. The present invention is effective even on these probes because an output value difference results from the distance from the position of the group of conversion elements 004 forming the transmission aperture or the reception aperture to the object surface on the image scan line 025.

A probe with conversion elements arranged on a bowl-shaped support member can receive, in various directions, acoustic waves propagating from the object, improving the accuracy of reconstructed images. In the bowl-shaped probe, the conversion elements fail to have the same high-sensitivity direction. Consequently, when the holding member is partitioned into certain regions, the positions of the regions are precluded from being specified in association with the high-sensitivity directions of the conversion elements. On the other hand, the bowl-shaped probe is provided with a high-sensitivity area (high-resolution area) where the high-sensitivity directions of a plurality of elements concentrate. Thus, when positions in the holding member are identified, the positions can be specified in association with the high-sensitivity area. When the bowl-shaped probe is applied to a photoacoustic apparatus described below, a light irradiator is preferably provided at the bottom of the bowl.

In the present invention, the shape of the probe includes the protruding portion as described above. Thus, when the degree of attenuation of acoustic waves changes according to the position of the probe moving on a scanning region, the acoustic attenuation characteristics of the holding member are changed according to the degree of attenuation. This enables a reduction in variation in the intensity of acoustic signals and in the output value of image data.

### EMBODIMENT 2

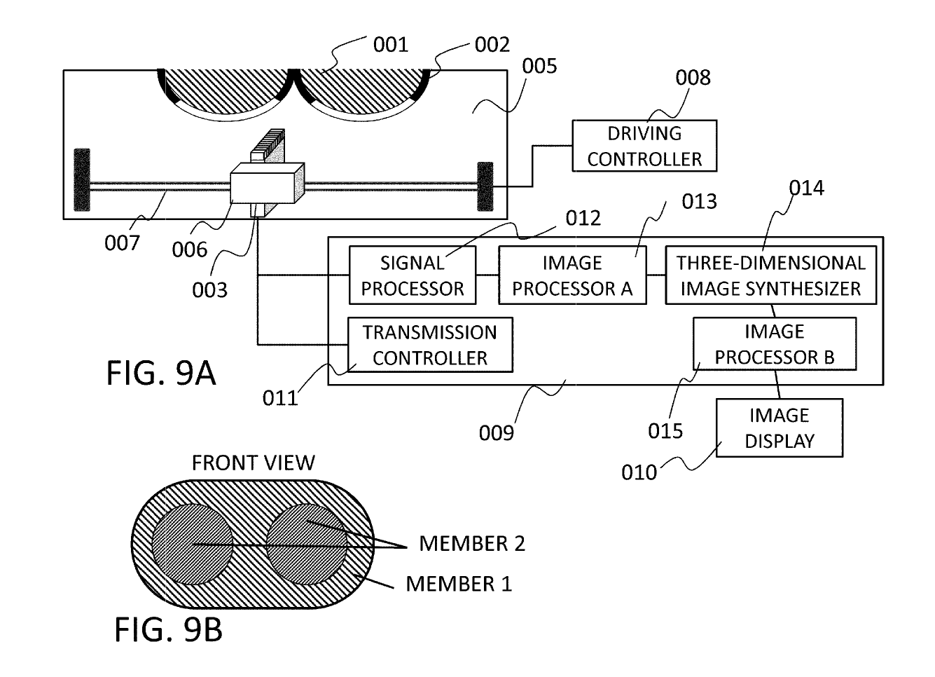

FIG. 9A depicts a schematic system diagram of an object information acquiring apparatus in the present embodiment. Components of the present embodiment that are the same as the corresponding components of the above-described embodiment are denoted by the same reference numerals and will not be described in detail. In the present embodiment, both the right and left breasts are selected as the object 001 and simultaneously imaged. Thus, Embodiment 2 is different from Embodiment 1 particularly in the structure of the holding member 002.

### Holding Member and Acoustic matching Material

Two different resin materials are used for the holding member 002 that holds the object 001. FIG. 9B depicts the holding member 002 as viewed from the probe scanning plane. A material for a member 1 is PETG, which is a type of PET. A material for a member 2 is polymethyl pentene. Each of the members is approximately 2.5 mm in thickness. The members are processed into desired shapes by compression molding and ejection molding and then joined together with an adhesive to form the holding member 002. The holding member 002 is sized to have a major diameter of 500 mm and a minor diameter of 250 mm and processed such that the distance between the chest wall and the nipple pair is approximately 35 mm.

For the acoustic attenuation characteristics of the members, the degree of attenuation is approximately 1.2 [dB/MHz/cm] for polymethyl pentene and approximately 4 [dB/MHz/cm] for PETG. The object 001 and the holding member 002 are in close contact with each other in order to eliminate the gap between the object 001 and the holding member 002 as much as possible.

The acoustic matching material 005 is water and was used while being circulated by a pump. In the present embodiment, water temperature was kept at approximately 35° C. using a heater. Keeping the water temperature in this manner is effective for preventing the subject from feeling uncomfortable and for defining a sound velocity in the acoustic matching material 005 to improve the accuracy of image reconstruction.

### Miscellaneous Apparatus Components

In the present embodiment, the probe 003 is a 1D linear probe with 256 channels. The conversion elements 004 forming the probe 003 are PZTs having a central frequency of 8 MHz and an element size of 4 mm and arranged at a lateral element pitch of 0.2 mm. Methods by implemented by the system controller 009 for control of transmitted ultrasonic waves, reception of echo waves, processing of reception signals, an image reconstruction process, and the like in Embodiment 2 are similar to those in Embodiment 1.

The probe 003 installed on the carriage 006 transmits and receives ultrasonic waves while moving through a planar scanning region, in accordance with instructions from the driving controller 008. In the present embodiment, the carriage 006 is moved at any speed to any position in a biaxial direction by the driving mechanism 007 with a combination of a pulse motor and a ball screw. In the present embodiment, for three-dimensional image data resulting from a reception signal, any desired sectional image can be checked on the image display 010, which is a liquid crystal display. If the desired sectional image depicts a marked output value difference, the output values can be adjusted using the image processor B (015).

The above-described system was used to acquire images of the breasts and to display a C plane image of a position close to the chest wall. Compared to a C plane image of the same position obtained when the holding member 002 was not used, the C plane image obtained using the holding member 002 was vivid to see due to improvement in the difference in image output value between the peripheral portion of each of the breasts and a position close to the nipple. The inventors found that the quality of the image of any desired section was prevented from being degraded. The system in the present embodiment can be applied not only to the right and left breasts but also to objects such as human buttocks and a human foot with an arched portion which are depressed at the central portion and protrude at the peripheral portion and which are depressed again at the outermost peripheral portion.

### EMBODIMENT 3

The present embodiment is characterized in that the measurement target is an object such as one of the breasts which protrudes toward the probe at the central portion of the object. Therefore, the apparatus in the present embodiment is different from the apparatus in Embodiment 2 in that the holding member 002 is shaped as depicted in FIG. 6 for Embodiment 1. Specifically, the holding member has such a generally concentrically circular acoustic attenuation characteristics distribution as exhibits acoustic attenuation characteristics the level of which is lowest at the central portion of the holding member and increases toward the peripheral portion of the holding member.

Specifically, to form the holding member 002 that holds the object 001, two different resin materials were combined together as is the case with FIG. 7A. As in the case of Embodiment 2, PETG was used for the member 1 in the peripheral portion, and polymethyl pentene was used for the member 2 in the central portion. Each of the members is approximately 2.5 mm in thickness. The members were shaped by compression molding and ejection molding and then joined together with an adhesive. The holding member 002 was φ250 mm in size. The distance between the chest wall and the nipple was set to approximately 35 mm. The acoustic attenuation characteristics of the holding member 002 are as illustrated in Embodiment 2. The gap between the object 001 and the holding member 002 is eliminated wherever possible to make the object 001 and the holding member 002 in close contact with each other.

The above-described system was used to acquire images of the breasts and to display a C plane image of a position close to the chest wall. Compared to a C plane image of the same position obtained when the holding member 002 was not used, the C plane image obtained using the holding member 002 was vivid to see due to improvement in the difference in image output value between an image generated at the central portion of the object and an image generated at the peripheral portion of the object.

Consequently, when the object 001 is imaged which protrudes toward the probe 003 at the central portion of the object 001, possible degradation of the quality of an image of any desired section can be reduced by using the holding member 002 exhibiting a lower level of acoustic attenuation characteristics at the central portion than at the peripheral portion.

### EMBODIMENT 4

In the present invention, the measurement target is an object protruding toward the probe at the central portion of the object, as in the case of Embodiment 3. Therefore, the apparatus in the present embodiment uses the holding member 002 having such a generally concentrically circular acoustic attenuation characteristics distribution as exhibits acoustic attenuation characteristics the level of which is lowest at the central portion of the holding member and increases toward the peripheral portion of the holding member, as in the case of Embodiment 3. The present embodiment is different from Embodiment 3 in that the holding member 002 is formed of one member.

Specifically, as the holding member 002 that holds the object 001, a sheet of different thickness was used for each segment as is the case with FIG. 4. A material for the sheet is natural rubber. The holding member 002 is suitably selected in accordance with the constitution of the subject. In the present embodiment, the subject is a middle-aged woman with a D cup. The sheet has the smallest thickness of 0.3 mm at the central portion thereof (corresponding to Pos1 in FIG. 4). The sheet has the largest thickness of 4.5 mm at the central portion thereof (corresponding to Pos2 in FIG. 4). The sheet is formed such that the film thickness gradually varies between Pos1 and Pos2. The sheet is shaped like a circle of φ250 mm. The holding member is subjected to an initial tension when installed in equipment such that the distance between the chest wall and the nipple is approximately 40 mm while the object is held. The holding member 002 in the present embodiment was produced by compression molding such that the difference in acoustic attenuation characteristics between Pos1 and Pos2 was about 1.7 dB/MHz.

The above-described system was used to acquire images of the breasts and to display a C plane image of a position close to the chest wall. Compared to a C plane image of the same position obtained when the holding member 002 was not used, the C plane image obtained using the holding member 002 was vivid to see due to improvement in the difference in image output value between an image generated at the central portion of the object and an image generated at the peripheral portion of the object.

Consequently, when the object 001 was imaged which protrudes toward the probe 003 at the central portion of the object 001, possible degradation of the quality of an image of any desired section was successfully reduced by using the holding member 002 formed of the single member and which had a smaller film thickness at the central portion than at the peripheral portion.

### EMBODIMENT 5

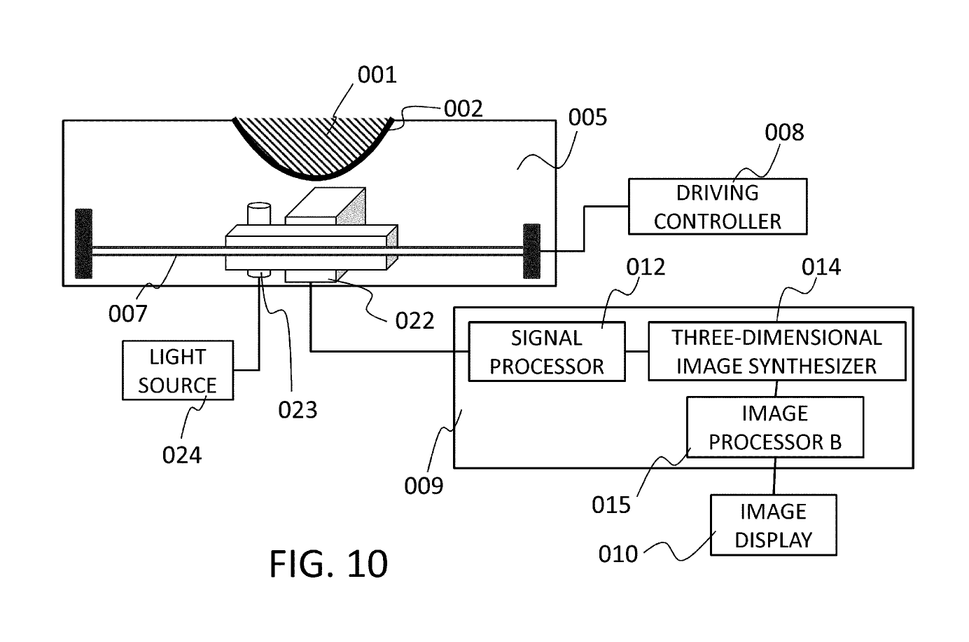

An object information acquiring apparatus in the present embodiment performs photoacoustic measurement to acquire characteristics information on the interior of the object. This will be described using FIG. 10. A probe 022 for photoacoustic waves and a light irradiator 023 that transmits light emitted from a light source 024 are attached to the carriage 006.

The light source 024 in the present embodiment is a titanium sapphire laser that is a type of solid laser and irradiates the object with pulsed light. Pulse interval was set to 10 Hz. As the laser light source, a gas laser, a pigment laser, a semiconductor laser, and the like may be utilized besides the solid laser. A flash lamp, a light emitting diode, or the like may also be utilized. As irradiation light, near infrared light is preferable. Wavelength is suitably approximately 650 to 1100 nm and was set to 750 nm in the present embodiment. To determine the concentrations of components of the object, a wavelength variable laser is preferably used which can radiate light with a plurality of wavelengths. Optical members such as bundle fibers, lenses, mirrors, or prisms were used to guide light from the light source to the light irradiator.

When an optical absorber inside the object absorbs the energy of radiated light, the optical absorber is thermally expanded to generate an acoustic wave. Examples of the optical absorber having characteristics of absorbing near infrared light include in vivo blood containing a large amount of hemoglobin and blood vessels and tumor tissues containing a large amount of new blood vessels.

In the present embodiment, the light irradiator 023 was installed on the carriage 006 so as to be moved along with the probe 022 to efficiently irradiate an imaging portion with light. However, the installation location is not limited to this. The intensity of photoacoustic waves generated varies according to the amount of light reaching the imaging portion. Consequently, even for blood vessels in the same form, the intensity of reception signals varies according to the depth in the object. Thus, the system controller 009 in the present embodiment acquires the amount of light distributed in the object through measurement and calculation and uses the amount to correct the signal intensity. The amount of light radiated to the object is preferably controlled by adjusting the light intensity or the carriage position.

The probe 022 for photoacoustic waves is configured by arranging 600 1-mm×1-mm conversion elements 004 in a 20×30 array. The conversion elements 004 are PZTs with a central frequency of 2 MHz. The present embodiment is the same as the above-described embodiments in the techniques for receiving photoacoustic waves using the reception aperture and for executing signal processing and image reconstruction. The present embodiment also the same as the above-described embodiments in the adjustment of the output values during the image reconstruction process and after output of images to the display.

The holding member 002 in any of the above-described embodiments can be applied to the present embodiment. That is, a holding member can be utilized which is made of the same material and is provided with an acoustic attenuation characteristics distribution by varying the thickness of the holding member with segment or a holding member can be utilized which contains different materials in the respective segments. The holding member 002 in the present embodiment is transmissive not only for acoustic waves but also for light.

The above-described system was used to acquire images of the breasts and to display a C plane image of a position close to the chest wall. Compared to a C plane image of the same position obtained when the holding member 002 was not used, the C plane image obtained using the holding member 002 was vivid to see due to a reduction in the difference in image output value between an image generated at the central portion of the breast and an image generated at the peripheral portion of the breast, although there were some differences in beneficial results depending on the forms of holding member 002. Consequently, when the acoustic attenuation characteristics of the holding member 002 is distributed, the apparatus using the photoacoustic effect also enables a reduction in degradation of image quality resulting from the difference among the output values from any desired section.

### Variations

In the present invention, the holding member 002 that contacts and holds the object to define the shape of the object is not an essential component. The object of the present invention can be accomplished by varying the acoustic attenuation characteristics with position in the probe scanning plane according to the unevenness of the object. Thus, instead of the holding member that contacts the object, an acoustic attenuation member of a different acoustic wave attenuation rate for each segment may be provided at a position located away from the object and between the probe 003 and the object. The holding member in each of the embodiments of the present invention may also be referred to as an acoustic attenuator having an acoustic attenuation effect.

For example, for an object such as one of the breasts which protrudes at the center thereof, the acoustic attenuation member used has an attenuation rate that is low at a position opposite to a protruding portion of the object and that is high at a position opposite to a peripheral portion of the object. As such an acoustic attenuation member, a generally planar sheet of a different thickness for each position on the sheet may be utilized. Alternatively, a generally planar sheet may be utilized which is formed of a combination of raw materials with different acoustic attenuation characteristics. The present variation also enables reduction of the difference in brightness in the C plane and improvement of the accuracy of object images.

Alternatively, instead of the acoustic attenuation member that does not contact the object, an acoustic attenuation member may be provided which contacts the object depending on the size of the breast or the like. In that case, the acoustic attenuation member may be a material that is rigid enough to firmly support the object or a member that is elastically deformed upon coming into contact with the object.

According to an aspect of the present invention, an object information acquiring apparatus is provided which performs ultrasonic measurement or photoacoustic measurement on an uneven object while allowing a probe to scan the object in an imaging region and which enables a reduction in output value difference resulting from a variation in the distance from the probe scanning plane to any desired section of the object. This allows suppression of loss of information in images that is indicative of characteristics information on the object and also allows brightness unevenness to be restrained.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-079845, filed on Apr. 9, 2015, which is hereby incorporated by reference herein in its entirety.

What is claimed is:

1. An object information acquiring apparatus comprising:
a receiver in which a plurality of elements is arranged, each of the plurality of elements receives an acoustic wave propagating from an object and outputs an electric signal;
a scanner configured to move the receiver through a predetermined scanning region;
an information processor configured to acquire characteristics information on an interior of the object using the electric signal; and
an acoustic attenuator configured to be arranged between the receiver and the object in the scanning region and have an acoustic attenuation characteristics distribution corresponding to a shape of the object.

2. The object information acquiring apparatus according to claim 1, wherein the acoustic attenuator has an acoustic attenuation characteristics distribution that has a reduced degree of acoustic attenuation at a protruding portion of the object as viewed from the receiver.

3. The object information acquiring apparatus according to claim 1, wherein the acoustic attenuator has an acoustic attenuation characteristics distribution that has an increased degree of acoustic attenuation at a depressed portion of the object as viewed from the receiver.

4. The object information acquiring apparatus according to claim 1, wherein the scanning region through which the scanner moves the receiver is a planar scanning plane.

5. The object information acquiring apparatus according to claim 4, wherein the acoustic attenuator has an acoustic attenuation characteristics distribution whose degree of acoustic attenuation increases as a distance from the scanning plane to the acoustic attenuator increases in a direction normal to the scanning plane.

6. The object information acquiring apparatus according to claim 4, further comprising acoustic matching material configured to acoustically match the receiver with the object.

7. The object information acquiring apparatus according to claim 6, wherein
the information processor acquires characteristics information on a plane, which is parallel to the scanning plane, in the object, and
the acoustic attenuator has an acoustic attenuation characteristics distribution corresponding to a ratio between a distance, over which the acoustic wave passes through the acoustic matching material, and a distance, over which the acoustic wave passes through the interior of the object, in a path from the receiver to the plane in the direction normal to the scanning plane.

8. The object information acquiring apparatus according to claim 1, wherein the acoustic attenuator is thicker at a portion thereof corresponding to a depressed portion of the object as viewed from the receiver than at a portion thereof corresponding to the protruding portion of the object as viewed from the receiver.

9. The object information acquiring apparatus according to claim 1, wherein the acoustic attenuator is formed by laying a plurality of sheet members on top of one another, and more sheets are laid on top of one another at a portion of the acoustic attenuator corresponding to a depressed portion of the object as viewed from the receiver than at a portion of the acoustic attenuator corresponding to the protruding portion of the object as viewed from the receiver.

10. The object information acquiring apparatus according to claim 1, wherein the acoustic attenuator is formed by combining a plurality of members together, and members having a higher degree of acoustic attenuation are provided at a portion of the acoustic attenuator corresponding to a depressed portion of the object as viewed from the receiver than at a portion of the acoustic attenuator corresponding to the protruding portion of the object as viewed from the receiver.

11. The object information acquiring apparatus according to claim 1, wherein the acoustic attenuator is a holding member that holds the object.

12. The object information acquiring apparatus according to claim 11, wherein the holding member holds a breast as the object, and a difference in acoustic attenuation characteristics between a central portion and a peripheral portion of the holding member is 0.3 to 7.2 [dB/MHz].
